(19) United States
(12) Patent Application Publication
(10) Pub. No.: US 2006/0147432 A1
(43) Pub. Date: Jul. 6, 2006

(54) Tolerogenic antigen-presenting cells

(75) Inventors: Marilyn Jean Moore, (US); Andrew James Leishman, Loughborough (GB); Donald Alan James Innes, Edinburgh (GB)

Correspondence Address: KING & SPALDING LLP, 1180 PEACHTREE STREET, ATLANTA, GA, 30309, US

(73) Assignee: Revivicor, Inc., Blacksburg, VA (US)

(21) Appl. No.: 11/361,647

(22) Filed: Feb. 24, 2006

Related U.S. Application Data
(63) Continuation of application No. 10/402,442, filed on Mar. 28, 2003, now abandoned.

(30) Foreign Application Priority Data
Mar. 28, 2002 (GB) 0207440.9

(51) Int. Cl. C12N 5/08; A61K 35/14

(52) U.S. Cl. 424/93.7; 435/372

(57) ABSTRACT

It has been found that dendritic cells can be prepared which cannot mature. These cells can provide signal 1 to T cells but cannot provide co-stimulatory signal 2. T cells which are stimulated by the permanently immature dendritic cells therefore anergise, so the dendritic cells are tolerogenic rather than immunogenic. The cells are generally CD40^{-ve}, CD80^{-ve} and CD86^{-ve}, and remain so when stimulated by inflammatory mediators such as lipopolysaccharide. The cells can be prepared conveniently by the culturing adherent embryonic stem cells in the presence of GM-CSF.

This application claims priority to GB 0207440.9, filed on Mar. 28, 2002.

### TECHNICAL FIELD

The invention is in the field of transplantation. In particular, it is in the field of preventing transplant rejection. It achieves this by administering to a transplant recipient antigen-presenting cells which tolerise anti-graft T cells.

### BACKGROUND ART

The mammalian immune system plays a central role in protecting individuals from infectious agents and preventing tumour growth. However, the same immune system can produce undesirable effects such as the rejection of cell, tissue and organ transplants from unrelated donors. Furthermore, the immune system can malfunction and lead to the destruction of an individual's own tissue in a process known as autoimmunity.

Immunosuppressive drugs have offered a solution to the problem of adverse immune responses, but they do not selectively target the response in question. Use of such drugs leads to systemic suppression of both appropriate and undesirable responses and can lead to failures in the control of infection and tumours. However, as the functional mechanisms underlying the immune response have become better understood, the specific elimination of undesirable immune responses has become a goal in medicine [1].

In many ways the immune response is controlled by T lymphocytes (T cells) and these have become the target for the induction of immunological non-responsiveness or tolerance [2]. A range of surface molecules found on T cells have been targeted with their natural ligands or synthetic peptides from these ligands, and effects on T cell responsiveness observed [3, 4]. However, these function like immunosuppressive drugs and do not target specific T cells without further intervention.

It is clear that T cell responses are normally tightly controlled in vivo, and it is thought that another cell population is most likely to carry out this control function. The dendritic cell (DC) has been extensively studied in this context [5-9]. DCs are acknowledged as having one of the most important roles in many immune responses, being uniquely able to both stimulate and tolerise T cells. DCs can pick up and process antigens via endocytosis (macropinocytosis, phagocytosis and clathrin-mediated endocytosis) to present peptides from these antigens in the context of major histocompatability complex (MHC) to T cells [10]. When a T cell receptor (TCR) recognises its specific peptide on MHC this is known as signal 1. This signal alone is insufficient to activate T cells and, when supplied in isolation, has been shown to tolerise them by inducing anergy. In the presence of inflammatory stimuli, DCs can mature and upregulate co-stimulatory molecules on their surface which interact with their ligands on the surface of the T cells, thus providing signal 2, which will activate the T cells. However, the exact characteristics that determine whether a DC is activating or tolerogenic are currently being elucidated.

One determining characteristic seems to be be the state of maturation of the DC. Whereas mature DC have all the surface molecules required to activate the T cells in that they can present antigen to the TCR as well as provide the necessary costimulatory/activating signals, immature DCs only have the antigen presenting molecules on their surface, usually at low levels. Thus, immature DCs cannot activate T cells [11]. However, maturation state is not always a reliable indicator of immunogenicity as DCs with a mature phenotype have been shown to induce T cells to undergo activation induced cell death [12] and thus induce tolerance.

The function of DCs in immune regulation has also been explained in terms of diverse DC subsets and lineages. Phenotype markers and function of the DCs have been used to separate DCs into different groups [13] e.g. myeloid and lymphoid DCs. Examples of both immunogenic and tolerogenic DCs have been described in each subset [14].

While the precise characteristics and phenotype of tolerogenic DCs are unclear, there have been several attempts to use various types of DC in tolerance induction.

The production of immature DCs derived from precursors in peripheral blood mononuclear cells (PBMCs) which can be used to induce tolerance is described in references 15 and 16. However, there are drawbacks to this method. In particular, these DCs could be matured under conventional conditions into fully immunogenic cells. The chance of maturation in vivo is therefore high, particularly at sites of inflammation in the recipient. Furthermore, genetic manipulation of primary cells is difficult, and that are also likely to mature into fully immunogenic cells. Also, as the tolerogenic cells must be matched to the donor tissue, this method of inducing tolerance requires the DCs to be made from precursors in the PBMCs of each individual donor, which would be costly.

A key objective in deriving cells for tolerance induction to transplants is to have them matched to the donor tissue. Embryonic stem (ES) cells are able to differentiate into a variety of cells and tissues, so ES cells could be differentiated into cells for transplantation and also into donor-matched tolerogenic cells. Thus, DC precursors from stem cells can be manipulated to produce DCs which are either tolerogenic or immunogenic. Methods for producing DCs from mouse ES cells are described in references 17 and 18. These methods result in the production of immunostimulatory DCs that can be matured by culturing with lipopolysaccaride in vitro. Indeed, these ES cell derived dendritic cells induce strong allograft responses from purified T cells to other cells of the same haplotype as the DCs. Thus, these DCs are not useful for inducing tolerance towards an allograft.

A further method of inducing antigen-specific tolerance is to halt maturation of antigen presenting cells, such as DCs, by using agonists of certain cell surface receptors [19, 20]. Since this method would require making tolerogenic APCs from each individual awaiting transplant or suffering from autoimmune disease, it would prove costly. Further, there is the possibility that the inhibition of maturation of the APCs could be reversed (e.g. when agonists are no longer supplied) which would have dire consequences for the patient as the tolerogenic APCs would become immunogenic and would thus make the graft rejection or autoimmunity worse.

A similar method is described in reference 21, but this method is based on the use of oligo-DNA decoys in order to sequester NF-KB. As mentioned above, however, this method is unsatisfactory because it is prone to reversal if the supply of oligo-DNA to DCs expires.

References 22 and 23 describe induction of tolerance to a graft using agents to inhibit DC maturation as well as reducing the recipient's T cell population by administering an immunotoxin. While this method may prove to be effective in reducing the immune response to the graft it may also have very dangerous consequences for the patient because it is not antigen-specific. Systemic immunosuppression would leave the patient very susceptible to secondary infections and cancer.

Finally, the use of TNFα and other inflammatory mediators to generate DCs from mononucleate cells derived from cytapheresis is described in references 24 and 25. However, as this method will likely produce immunogenic DCs it is unlikely to be useful for inducing transplantation tolerance.

It is an object of the invention to provide dendritic cells which are tolerogenic in a graft-specific (i.e. non-systemic) manner, which are inherently unable to present co-stimulatory signal 2 to a T cell, which are amenable to genetic manipulation, which are easily matched to graft tissue, which do not have to be matched to an individual patient, which are not prone to reversal to an immunogenic state, which are easily obtained, and which are non-tumorigenic.

### DISCLOSURE OF THE INVENTION

The inventors have found that it is possible to prepare an antigen-presenting cell (APC) which can present antigen to a T cell (thereby providing signal 1) but which is unable to provide co-stimulatory signal 2. The invention is based on the surprising finding that it is possible to prepare dendritic cells which cannot mature. These cells can provide signal 1 to T cells but cannot provide co-stimulatory signal 2. T cells which are stimulated by the permanently-immature dendritic cells therefore become anergic, and so the dendritic cells are tolerogenic rather than immunogenic. By providing a tolerogenic cell which matches the haplotype of graft tissue, anti-graft T cells are therefore removed.

Tolerogenic Cells of the Invention

The invention provides a dendritic cell which is immature and cannot mature.

Unlike natural immature dendritic cells, and in contrast to the dendritic cells described in references 11 and 17, the dendritic cells of the invention cannot mature when, for example, they are stimulated by inflammatory mediators such as lipopolysaccharide (LPS), tissue necrosis factor α (TNF-α), phytohemagglutinin (PHA), or conconavalin A (ConA). They are able to present antigens to T cells, thereby providing signal 1, but they cannot provide co-stimulatory signal 2 because they remain in an immature state.

The invention also provides a dendritic cell which can deliver signal 1 to a T cell (antigen presentation), but which cannot provide signal 2 to the T cell, either in a resting state or when stimulated by an inflammatory mediator.

The invention also provides a dendritic cell which: (a) is able to present antigens to T cells; (b) is CD40^{-ve}, CD80^{-ve} and CD86^{ve}, and (c) remains CD40^{ve}, CD80^{-ve} and CD86^{-ve} when stimulated by an inflammatory mediator.

CD40, CD80 and CD86 are co-stimulatory molecules. The cells of the invention are thus tolerogenic and non-immunogenic. They are able to induce T cell tolerance to allo-antigens in vitro and in vivo.

The cells are preferably MHC-II^{+ve}. Expression of MHC-II allows the cells to tolerise CD4 T cells (helper T cells), even at low levels. The cells may be MHC-I^{+ve} or MHC-I^{-ve}. MHC-I expression is only specifically necessary when it is desired to tolerise CD8 T cells (cytotoxic T cells). The precise MHC-I and MHC-II phenotype of a cell and the necessary levels of expression will depend on the type of tolerisation desired, but the overall requirement of the cells is that they can present antigens to T cells.

The cells of the invention are preferably CD34^{-ve} i.e. they are not haematopoetic stem cells.

The cells may be CD11c^{-ve}. CD11c is an integrin which is displayed on the surface of mature dendritic cells and which plays a role in binding to the iC3b protein of the complement cascade. CD11c^{-ve} cells cannot activate the complement cascade by binding to iC3b and so inflammatory responses are advantageously reduced.

The cells may be CD14^{-ve}. CD14 is the LPS receptor and so CD14^{-ve} cells will not be stimulated by this inflammatory mediator.

Cells of the invention may or may not have one of the following marker phenotypes: CD1d^{-ve}, CD54^{+ve}, CD95^{-ve}, CD11b^{+ve}, CD8α^{+ve}.

By “−ve” it is meant that the protein in question is not expressed at levels sufficiently high in a cell for its function to be manifested by that cell (e.g. a CD40^{-ve} cell does not manifest a CD40-mediated co-stimulatory phenotype). Expression may be wholly absent (e.g. as in genetic knockouts) but this is not always necessary, such as where expression is low enough (e.g. not be detectable above background or basal levels) for a protein's function not to be manifested. One way of measuring expression levels is by FACS assay, where “−ve” typically means that there is no significant signal difference between the, cells of the invention in the presence of anti-marker antibody (e.g. anti-CD40, anti-CD80, anti-CD86, etc.) and in the absence of the antibody (e.g. see FIG. 2).

Conversely, “+ve” means that the protein in question is expressed at levels in a cell such that its function is manifested by the cell (e.g. a T cell can interact with a MHC-II^{+ve} cell). The level of expression may be lower than, the same as, or higher than levels seen in wild-type dendritic cells. By FACS assay, “+ve” means that the presence/absence of anti-marker antibody gives a significant signal shift (e.g. ≧½ log).

The cells of the invention are preferably not immortal (i.e. they cannot propagate indefinitely in culture). The cells of the invention are preferably non-tumorigenic and may have a normal karyotype.

The cells of the invention are preferably human cells.

The cells of the invention may be clonal.

The cells of the invention can be myeloid or lymphoid dendritic cells.

The cells of the invention are preferably stable, in the sense that they will not revert to an undifferentiated state and will not further differentiate into immunogenic dendritic cells. Such changes would be dangerous as, rather than tolerising the recipient's immune system to a graft, the immunogenic cells would be primed and thus very quickly reject the transplanted tissue. Similarly, preferred cells are unable to revert to a maturable state and their tolerogenicity does not require the presence of exogenous molecules (e.g. agonists or oligo-DNA). This is a key advantage when compared to the dendritic cells of references 19, 20 and 21.

Thus the cells of the invention preferably do not comprise: (i) a single-stranded or double-stranded oligodeoxynucleotide (e.g. consisting of 25 or fewer nucleotides per strand) comprising one or more NF-KB binding sites; and/or (ii) an agonist of CD36, of CD51, or of a thrombospondin receptor.

The cells of the invention are preferably capable of endocytosis. They may also be capable of phagocytosis. It is preferred that the cells of the invention do not upregulate class II MHC expression during endocytosis or phagocytosis.

The cells of the invention can preferably survive in culture in vitro for at least four weeks (e.g. for at least 6 weeks, for at least 8 weeks, or for longer).

The cells of the invention are preferably differentiated in vitro from stem cells, such as ES cells. Thus the invention provides a tolerogenic dendritic cell differentiated in vitro from a stem cell (preferably from an ES cell).

Cells of the invention can be prepared in a number of ways. Most conveniently, they are prepared by the addition of appropriate growth factors to cause the differentiation of stem cells in culture, but they may also be prepared by preventing the functional expression of proteins which are crucial to dendritic cell maturation (e.g. by genetic manipulation, by antisense, by the use of antagonists etc.).

Differentiation Methods

The invention provides a process for preparing a tolerogenic antigen-presenting cell from a stem cell, wherein the method includes the step of culturing the stem cell in the presence of one or more cytokine(s) which cause(s) the stem cell to differentiate into the tolerogenic cell. The tolerogenic cells can then be recovered from culture medium.

The stem cell used in the process of the invention can be any multipotent or pluripotent stem cell, particularly one which can give rise to haematopoetic lineage. Pluripotent cells have the ability to develop into any cell derived from the three main germ cell layers. Adult stem cells, placental stem cells, fetal stem cells and umbilical stem cells may all be used, but preferred stem cells are ES cells. The invention includes the use of embryonic carcinoma (EC) cells or embryonic germ (EG) cells [e.g. 26].

Methods for obtaining these stem cells and for maintaining them (e.g. in an undifferentiated state) prior to use in the process of the invention are well known.

ES cells are cells isolated from embryos which can propagate indefinitely in in vitro culture. ES cells are pluripotent, that is they have the ability to give rise in vivo to all cell types which comprise the adult animal. Murine [e.g. ref. 27] and human [e.g. refs. 28 & 29] ES cells are readily available and conditions for their undifferentiated growth are well known [e.g. refs. 30 to 40]. Some ES cells are properly referred to as pluripotent rather than totipotent, as they are incapable of forming some cell types, notably trophoblast, but trophoblast formation from human ES cells has been reported [41].

Human stem cells, and human ES cells in particular, are preferred for use according to the invention, in order to ensure compatibility with humans patients. Where non-human patients are to be treated, however, stem cells from other organisms (e.g. from non-human primates or from mice) may be used. Non-human stem cells may also be used for human administration in conjunction with techniques used in xenotransplantation.

Although it has not yet reached the same levels as for murine ES cells, knowledge on the growth and differentiation of human ES cells is advanced [e.g. refs. 39 to 44], as is information about how to derive cells of hematopoietic lineages with the potential to induce tolerance from various progenitors such as from human hematopoietic stem cells [e.g. refs. 13 & 45 to 49].

The stem cell is preferably a human ES cell line which is eligible for US federal funding according to criteria outlined by President Bush in his address of 9th Aug. 2001. More preferably, the stem cell is one which can be obtained from the NIH Human Embryonic Stem Cell Registry.

The human ES cell may be HES-1 or HES-2 [50].

Prior to differentiation, ES cells are preferably maintained in an undifferentiated state in a medium containing a suitable inhibitory factor (e.g. leukaemia inhibitory factor (LIF) for murine ES cells).

Cells are preferably maintained in an undifferentiated state in pre-gelled flasks (e.g. with 0.1% gelatin). In this way, the method of the invention can avoid the use of feeder cells and so, unlike reference 18, it is preferred not to use a feeder layer during pre-differentiation ES cell culture.

Stem cells will generally be allowed to develop into embryoid bodies (EBs) before differentiation into tolerogenic cells. The EBs are not themselves tolerogenic. EBs are aggregates of cells which are formed when ES cells, EG cells or EC cells are grown in suspension culture (e.g. when plated on a non-adhesive surface that prevents cell attachment). They develop spontaneously in liquid suspension culture and this does not require the presence of any particular cytokines. EBs are widely recognised in the art and can be produced routinely [e.g. refs. 51 to 54] from both human [e.g. refs. 42 & 55 to 59] and mouse cells. If the starting stem cells are in adherent culture, they can be disengaged from a tissue culture surface prior to the formation of EBs by methods involving the use of mechanical disaggregation, enzymatic treatment (e.g. with trypsin, papain, collagenase etc.), and/or metal ion chelators (e.g. EDTA, EGTA) etc. For differentiation to proceed optimally, EBs are preferably free-floating.

During differentiation in the presence of cytokine(s), it is preferred that cells are (unlike the EBs) maintained in adherent culture (e.g. on a plastic surface). After adhering, the EBs give rise to colonies of stromal cells which migrate outwards. After culture for 7 to 10 days, tolerogenic cells of the invention develop around the periphery and these can be harvested with around 90% purity.

Unlike reference 18, it is preferred not to use a feeder layer during differentiation of the EBs. Pre-gelled flasks (e.g. with 0.1% gelatin) can be used instead. This advantageously avoids the presence of undefined factors in the culture medium.

The cytokine will typically be added to the medium in which EBs are being cultured or maintained. The cytokine is preferably granulocyte macrophage colony stimulating factor (GM-CSF). This may be used in combination with one or more further cytokine(s) (e.g. interleukin-3 (IL-3), TNF-α, FLT3-ligand), but none of these three further cytokines alone is sufficient to bring about the desired differentiation. The method of the invention may optionally be performed in the absence of IL-3, in the absence of TNF-α, and/or in the absence of FLT3-ligand. The culture medium preferably lacks compounds such as FLT-3 ligand (‘Flt3-L’) and TNF-α, both of which have previously been reported as favouring the production of maturable dendritic cells.

The concentration of GM-CSF in the culture medium will generally be in the range 5-100 ng/ml e.g., 10-50 ng/ml, 20-30 ng/ml, or around 25ng/ml. Addition of IL-3 at up to 6 ng/ml does not appear to affect the development of tolerogenic cells, but may slightly increase the yield of cells produced.

Various forms and derivatives of GM-CSF are available and can be used in the invention. For example, it can be purified from blood, it can be expressed recombinantly [e.g. 60, 61], or it can be purified from the culture supernatant of a cell which secretes GM-CSF. The cytokines may alternatively be provided by including cells in the culture medium which secrete them. The addition of purified recombinant cytokines to the culture medium is preferred.

Cytokines are preferably from the same species as the stem cells (e.g. use human GM-CSF with human stem cells).

The culture media may contain serum or may be serum-free. If serum-free medium is used, it is preferred to use a serum replacement instead.

Inhibition of Functional Expression of Maturation Proteins

The culture methods of the invention produce dendritic cells which are unable to mature. The same effect can be achieved by other methods to inhibit or prevent expression of functional signal 2 proteins such as CD40, CD80 (B7-1) and CD86 (B7-2), although the culture methods are preferred.

For example, expression of the genes encoding signal 2 proteins can be prevented. This may involve knockout mutations to remove or mutate of their coding and/or regulatory sequences. Suitable knockout mutations can be achieved using techniques such as gene targeting. Expression can also be prevented using antisense techniques [e.g. refs. 62 to 65 etc.] or RNA silencing using RNAi [e.g. refs. 66 to 69], although such techniques are not preferred due to their reversible nature.

As an alternative, the function of signal 2 proteins can be inhibited by mutating key amino acid residues [e.g. refs. 70, 71, 72 etc.].

These techniques may be used singly or in combination. For example, CD40 expression could be prevented by knockout mutation, CD80 expression could be prevented by antisense, and CD86 could be inhibited by mutation. In general, however, permanent prevention techniques are preferable.

Immunotherapeutic and Immunoprophylactic Methods

The invention provides a method of inhibiting graft rejection in a recipient, wherein dendritic cells of the invention are administered to the recipient.

The invention also provides dendritic cells of the invention for use as a medicament.

The invention also provides the use of dendritic cells of the invention in the manufacture of a medicament for inhibiting graft rejection in a recipient.

The cells of the invention may be administered to a patient in pure form or in combination with other types of cell. It is preferred, however, that they should not be administered with immortal cells, with stem cells and/or with dendritic cells which are mature or capable of maturing.

The cells of the invention may be administered to a patient together with other active agents, such as one or more anti-inflammatory agent(s), anti-coagulant(s) and/or human serum albumin (preferably recombinant), typically in the same injection.

The cells will generally be administered to the recipient by injection (e.g. into the blood). Intravenous injection is preferred. The hepatic portal vein is a preferred route. Thus the invention provides a syringe containing cells of the invention.

The cells will generally be administered to a patient essentially in the form in which they exit culture. In some cases, however, the cells may be treated between production and administration. For instance, the cells may be irradiated prior to administration e.g. to ensure that the cells cannot divide. The cells may be exposed to antigens of interest prior to administration. The cells may be preserved (e.g. cryopreserved) between production and administration.

The cells will be administered in an amount effective to enhance tolerance to a graft. The number of cells to be delivered to a patient is based on a number of parameters, including: the body weight of the recipient, the activity of their immune system, and the tolerogenic efficacy of the cells. A typical number of cells would be around 10⁶-10⁸ cells per kg body weight.

The cells will be delivered in combination with a pharmaceutical carrier. This carrier may comprise a cell culture medium which supports the cells' viability. The medium will generally be serum-free in order to avoid provoking an immune response in the recipient. The medium is preferably free from animal-derived products (e.g. BSA). The carrier will generally be buffered and/or pyrogen-free.

The invention provides a method for transplanting a graft into a recipient, wherein the method involves the administration of dendritic cells of the invention together with the graft. The invention also provides a method for enhancing tolerance in a graft recipient, comprising the administration of dendritic cells of the invention to the recipient.

The dendritic cells may be administered before the graft (i.e. pre-tolerisation) or at substantially the same time. It is preferred to administer the cells before the graft (e.g. at least 1 day before, preferably at least 3 days before, and typically at least 5, 6, 7, 8, 9 or 10 days before).

The invention also provides a method for maintaining tolerance to a graft, wherein the method involves the administration of dendritic cells of the invention to a patient who has received a graft. This provides a ‘booster’ tolerisation.

The invention also provides a kit comprising (a) a tolerogenic cell of the invention and (b) a tissue graft for transplanting into a recipient, wherein (a) and (b) have histocompatible haplotypes (e.g. HLA haplotypes).

The graft may be any tissue, organ or cell suitable for transplantation e.g. heart, lung, kidney, liver, pancreas, islets of Langerhans, pancreatic β-cells or other insulin-producing cells, cornea, cartilage, bone marrow, nervous tissue, etc. It may be taken from a donor or may have been grown in vitro. The graft is preferably grown in vitro from stem cells.

The dendritic cells will generally have a haplotype (e.g. a HLA haplotype) which is histocompatible with the graft. This allows the dendritic cells to tolerise the recipient only to antigens from the graft. This can be achieved conveniently by deriving the dendritic cells and the graft from the same stem cells. It can also be achieved by conventional HLA matching. If the dendritic cells are not matched to the graft then they will have to be pre-exposed to graft antigens. Matching is advantageous because it favours antigen presentation to T cells by the direct pathway rather than the indirect pathway.

It is preferred that the dendritic cells will have a haplotype substantially different from the recipient. This reduces the risk of the dendritic cells tolerising the recipient to non-self antigens which are harmful e.g. to viral antigens. However, as the difference between graft and recipient haplotype increases, so does the requirement for robust tolerisation by the dendritic cells of the invention. For any given patient, the ideal position is a compromise between these two competing requirements.

The cells of the invention may be pre-loaded with graft antigens.

It is preferred that the graft and the recipient are from the same species (i.e. allo-transplantation), but the invention may also be applied where the graft and the recipient are from different species (i.e. xeno-transplantation). Where xeno-transplantation is used, it may be desirable to administer to the recipient further anti-xeno-response agents. Immunosuppressive drugs could be administered, but preferably those which are compatible with tolerance induction (e.g. rapamycin, but not cyclosporin).

The dendritic cells and the graft are preferably from the same species as each other.

The tolerogenic dendritic cells of the invention can be used in vitro to induce allogeneic T cells to be tolerant (i.e. non-responsive) towards other cells of the same haplotype as the tolerogenic cells. This can be achieved by incubating the allogeneic T cells with the tolerogenic cells e.g. for 3 days or longer (e.g. at least 4, 5, 6, 7, 8 days or more). When these allogeneic T cells are separated from the tolerogenic cells (e.g. by washing, followed by resting overnight) they can be put in vitro with cells or tissues which have the same haplotype as the tolerogenic cells. Compared to allogeneic T cells that have not been previously exposed to any cell with the same haplotype as the tolerogenic cells or allogeneic T cells that have been exposed to cells with the same haplotype as the tolerogenic cells, these allogeneic T cells that were previously exposed to the tolerogenic cells are tolerant in that they do not proliferate significantly compared with the allogeneic cells from the other two scenarios.

Autoimmunity

As well as being useful in inhibiting graft rejection, the dendritic cells of the invention can be used in the treatment of autoimmune diseases by tolerising auto-reactive T cells.

The invention provides a method of inhibiting an autoimmune reaction in a patient, wherein dendritic cells of the invention are administered to the patient.

The invention also provides the use of dendritic cells of the invention in the manufacture of a medicament for inhibiting an autoimmune reaction.

The methods and means of administration are generally as described above for immunotherapeutic and immunoprophylactic methods. The main difference, however, is that the dendritic, cells will be derived from stem cells from the autoimmune patient.

Genetic Manipulation of Stem Cells for Use in the Process of the Invention

A stem cell may have been genetically manipulated prior to use in the process of the invention. Similarly, differentiated derivatives of the stem cells may be genetically manipulated after the process of the invention has been performed.

For instance, a cell may have been genetically manipulated to encode a polypeptide (e.g. a transcription factor) which promotes differentiation of the stem cell into a dendritic cell.

Expression of this polypeptide may be controlled so that it occurs in the stem cell itself, or so that it occurs in a derivative of the stem cell (e.g. in an embryoid body). This may involve activation of the endogenous genes and/or introduction of exogenous genes.

Similarly, a cell may have been genetically manipulated such that it under-expresses or does not express a polypeptide (e.g. a transcription factor) which either favours differentiation away from a tolerogenic phenotype or which inhibits the development of a tolerogenic phenotype. For instance, genes could be knocked out, or could be inhibited using antisense or RNA silencing techniques.

A cell may have been genetically manipulated to express or over-express surface proteins which down-regulate immune responses (e.g. Fas-Ligand, CTLA-4-Ligand or Notch-Ligand). This may further enhance the non-immunogenic nature of the dendritic cells.

A cell may have been genetically manipulated not to express or to under-express surface and/or secreted proteins which promote T cell activation, such as CD40, CD80 or CD86. This may further enhance the non-immunogenic nature of the dendritic cells. This will typically be by the use of knockout techniques, but various other methods for preventing the expression or activity of such genes are well documented [73].

A cell may have been genetically manipulated to include a “suicide gene”. This provides a method of selectively killing cells such as undifferentiated stem cells which may persist in cell preparations to be used for transplant therapy, or all cells (differentiated or undifferentiated) derived from the stem cells as a failsafe mechanism to destroy the cells after transplantation. Suicide genes encode protein products that have no appreciable direct effect on cellular function, but which are capable of conferring toxicity by their ability to convert otherwise non-toxic substances (frequently termed prodrugs) into toxic metabolites. Suicide gene technology has been developed as a means of rendering cancer cells more sensitive to chemotherapeutics and also as a safety feature of retroviral gene therapy. Several combinations of suicide genes and prodrugs are known in the art [e.g. ref. 74] and include: HSV thymidine kinase+ganciclovir or acyclovir; E. coli cytosine deaminase+5-fluorocytosine; E. coli nitroreductase+CB1954 etc. The suicide gene is preferably under the control of a promoter expressed in undifferentiated stem cells or in other cells undesirable for transplantation (e.g. tumors or tumorigenic cells), in which case undifferentiated cells can be removed from culture by using the appropriate prodrug without affecting differentiated cells. Suitable promoters include those of the genes encoding Oct3/4 [75], Oct6 [76], Rex-1 [77]. and Genesis [78] etc. For use as a failsafe mechanism to allow a selective killing of a transplant in a patient (e.g. where the transplant is found to be harmful in a recipient), however, the suicide gene will generally be under the control of a constitutive promoter, although tissue-specific or inducible promoters could also be used.

A cell may have been genetically manipulated to insert markers suitable for lineage selection, a technique which specifically selects a desired cell type e.g. based on a previously-inserted recombinant construct which comprises a tissue-specific promoter linked to a selectable marker. Suitable gene promoters include, but are not restricted to, developmentally important factors (e.g. CD11b) and proteins characteristic of dendritic cells (e.g. CD83). Suitable selectable marker genes include, but are not restricted to, drug selectable genes (e.g. the G418 resistance gene neo, hygro, puro, zeo, bsd, HPRT), visible markers such as fluorescent proteins (e.g. GFP, DsRed) and genes which facilitate selection by automated cell sorting (e.g. genes. encoding cell surface antigens).

The stem cell may have been genetically manipulated to encode an antigen against which tolerance is desired. The antigen will be expressed, processed and presented and the tolerogenic cells of the invention will therefore anergise T cells which recognise this antigen.

The genetic manipulations described above may be used singly, or two or more may be used in combination.

Genetic manipulation of the stem cell may occur through random integration into the genome or, preferably, by gene targeting. As an alternative the manipulation may, where appropriate, use an episomally-maintained vector (e.g. a plasmid). Transfection of ES cells, including human ES cells [59], is well known.

For random integration, vector(s) which encode the relevant polypeptides may be introduced into the stem, cell. Typically, expression would be achieved using an expression vector comprising a gene promoter operably linked to DNA encoding the relevant polypeptide. DNA encoding the polypeptide may be cDNA, genomic sequences or a mixture of both. The promoter may direct constitutive or inducible expression and may be tissue-specific. Examples of constitutive promoters include the promoters from phosphoglycerate kinase (PGK), elongation factor 1α (EF1α), β-actin, or SV40. Examples of inducible gene promoters include systems composed of a chimeric transactivator that reversibly binds to the promoter region of the expression construct in response to a drug or ligand (e.g. mifepristone, tetracycline, doxycycline, ecdysone, FK1012, or rapamycin). The promoter is preferably derived from the PGK gene.

An alternative to the addition of recombinant constructs by random integration into the genome is the precise alteration of genes in situ by homologous recombination, termed “gene targeting”. This is the precise predetermined modification of genes by homologous recombination between introduced DNA and chromosomal DNA. Gene targeting can be used to insert, replace, rearrange or remove chosen DNA sequences in cultured cells, most commonly embryonic stem cells [e.g. ref. 79]. In some circumstances gene targeting may be preferable to simple introduction of an expression vector at a random site because the genetic modification can be predetermined to avoid any deleterious effect (e.g. oncogenic transformation) that would reduce the therapeutic value of derived cells.

Gene targeting may be used to achieve constitutive or inducible expression of a gene of interest by modifying or replacing the natural promoter or other regulatory regions of that gene. For example, a gene promoter may by replaced with a constitutive or inducible promoter (e.g. PGK) or elements which direct constitutive expression may added adjacent to the endogenous gene promoter. Methods to achieve such modifications by gene targeting, including in ES cells, are well known in the art.

It is also possible to perform genetic manipulation on a cell other than a stem cell, and then to transfer that genetic manipulation into a stem cell (e.g. by transfer of a nucleus into an enucleated stem cell) or into an embryo (e.g. by transfer of a nucleus into an enucleated oocyte) which can give rise to a stem cell. Both of these approaches indirectly give a genetically-manipulated stem cell.

Screening Assays

The cells of the invention may be compared to wild-type cells in order to identify factors involved in the maturation of dendritic cells. For instance, the mRNA populations of the two cells can be analysed using nucleic acid arrays.

### BRIEF DESCRIPTION OF THE DRAWINGS

FIG. 1 shows a phase contrast image of ES cell-derived tolerogenic cells of the invention. The cells are clusters of tolerogenic cells 10 days after putting EBs into 6-well plates with GM-CSF and IL-3.

FIG. 2 shows FACS analysis of the phenotype of tolerogenic cells of the invention using monoclonal antibody staining for surface expression of various cell markers.

FIG. 3 shows the inability of tolerogenic cells of the invention to mature. Expression levels of MHC-II and B7-2 (CD86) as measured by FACS analysis after incubation of tolerogenic cells with LPS or TNFα are shown.

FIG. 4 shows the ability of tolerogenic cells of the invention to tolerise allogeneic T cells in a two-step assay.

### MODES FOR CARRYING OUT THE INVENTION

1) Derivation and Maintenance of ES Cells from 129/P2 Mice

HM-1 murine embryonic stem cells were obtained from the 129/P2 mouse strain [80]. Tissue culture flasks were pre-coated with 0.1% gelatin in PBS to promote adherence of the HM-1 cells and they were maintained in Complete Medium (BHK-21 media supplemented with 10% heat-inactivated fetal calf serum (FCS), 1 mM sodium pyruvate, 2 mM L-glutamine, 2 mM non-essential amino acids and 50 μM 2-mercaptoethanol). In order to keep the cells in an undifferentiated state, leukaemia inhibitory factor (LIF) was added to the media. Cells were kept in incubators at 37° C. with 5% CO₂.

2) Generation of Tolerogenic Cells from HM-1

When a T25 flask of undifferentiated HM-1 cells were confluent, they were trypsinised lightly, so clumps of cells appeared as opposed to all single cells, washed at 900 rpm for 2 minutes to allow clumps of cells to collect at bottom of tube, supernatant carefully removed and clumps gently resuspended in 5 ml Complete Medium without LIF. 1.5-2×10⁵ cells/cell clumps were plated onto 90 mm bacteriological plastic dishes in 10 ml Complete Medium. Under these conditions, the HM-1 cells failed to adhere to the bacteriological plastic but remained in suspension where they continued to proliferate and form embryoid bodies. The EBs became macroscopic spheres by day 4 of culture and adopted a cystic appearance by day 10-12. Cells were kept in incubators at 37° C. with 5% CO₂.

At day 4 the EBs were transferred to a universal tube and 60-80 μl were added to each well of 6-well, tissue culture plates. 2 ml/well of Complete Medium supplemented with 25 ng/ml recombinant murine GM CSF and 1000 U/ml recombinant murine IL-3 was added. Cells were kept in incubators at 37° C. with 5% CO₂.

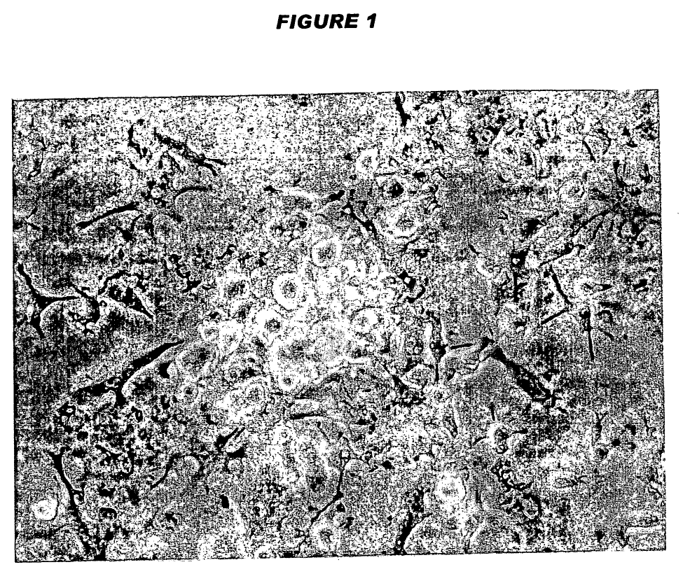

Within 24 hours of culture the EBs adhere to the plastic, and growth of differentiating cells, mainly stromal cells, emigrating outwards in a radial fashion appeared. Clusters of tolerogenic cells started to appear by day 4-5 and by day 8-10 the clusters were large enough to harvest tolerogenic cells (FIG. 1). Some of the tolerogenic cells adhered strongly to the plastic but most of them were lightly, adhered to the underlying layer of EB-derived stromal cells. They could be harvested by gentle pipetting and passaged over a 70 μm cell strainer to remove unwanted debris. Since the stromal layer which supports the generation of the tolerogenic cells is left intact, repeated harvesting of tolerogenic cells can be continued for 4 to 5 weeks.

3) Generation of Tolerogenic Cells from HM-1 without IL-3

EBs were generated and seeded onto 6 well plates as above, but IL-3 was not added to the medium. The generation of the tolerogenic cells in medium with GM-CSF (no IL3) occurred at essentially the same rate as medium with GM-CSF and IL-3. There was no detectable difference in the phenotypes of the GM-CSF and GM-CSF/IL3 populations.

4) Further Cytokines

As described above, tolerogenic dendritic cells could be obtained by culturing ES cells in the presence of GM-CSF, optionally combined with IL-3. Other recombinant cytokines (TNF-α & Flt3-L) were tested singly or in combinations and results were as follows:

| Cytokine(s) | Result |
|---|---|
| GM-CSF | + |
| GM-CSF + IL3 | + |
| GM-CSF + Flt3-L | + |
| GM-CSF + TNF-α | + |
| IL-3 | − |
| Flt3-L | − |
| TNF-α | − |

5) Characterization of ES-cell Derived Tolerogenic Cells

5.1 ) Phenotype

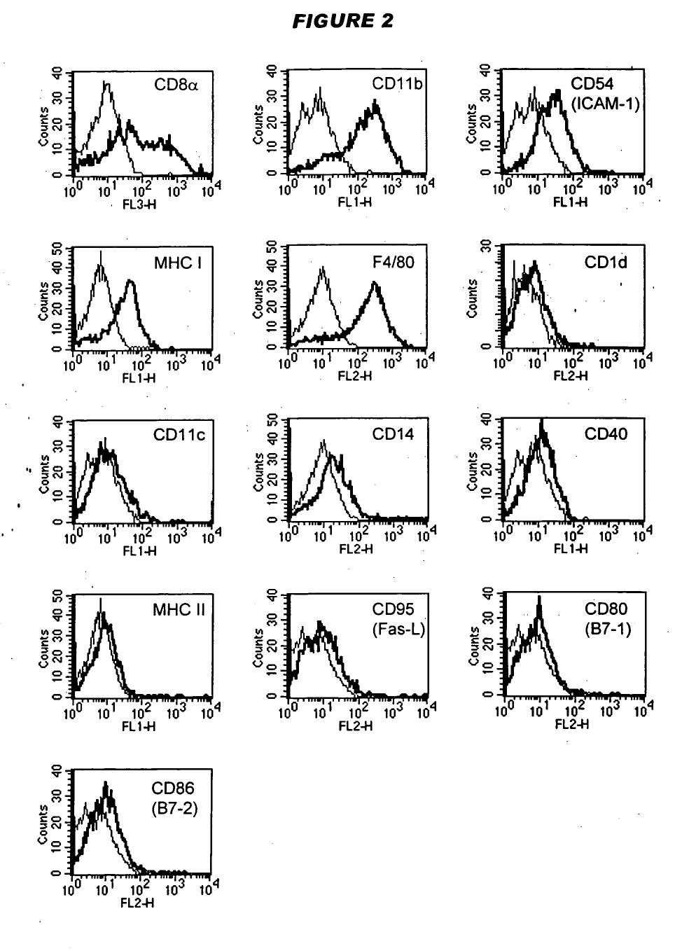

Tolerogenic cells were derived from ES cells as described above and analysed by flow cytometry for expression of surface markers using a panel of monoclonal antibodies (FIG. 2). CD8α, CD11b, CD54 (ICAM-1), MHC Class I and F4/80 were expressed at high levels on the surface of the tolerogenic cells. Low or insignificant expression of CD1d, CD11c, CD14, CD40, class II MHC, CD95 (Fas-Ligand), CD80 (B7-1) and CD86 (B7-2) was observed on the tolerogenic cells. CD11c is regarded as a mature dendritic cell-specific marker but under no circumstances was any significant expression of this molecule seen. The high expression of F4/80 suggests that the cells of the invention are similar to macrophages, but the morphology and adherent properties show that they are not macrophages. The low/insignificant level of expression of B7-1, B7-2, CD40 and MHC Class II suggests the cell is an immature dendritic cell.

By the identification methods used herein, therefore, the cells of the invention are classified as immature dendritic cells.

5.2) Activity

To further characterise the tolerogenic cells, their ability to phagocytose and endocytose was tested. The cells were prepared from EBs as described above. The cells were washed in Complete RPMI (i.e. RPMI supplemented with 10% heat-inactivated FCS, 1 mM sodium pyruvate, 2 mM L-glutamine, 2 mM non-essential amino acids and 50 μM 2-mercaptoethanol). Cells were resuspended in Complete RPMI with or without either FITC-labelled latex beads (to measure phagocytosis) or FITC-dextran (to measure pinocytosis) and kept at 4° C. or 37° C. for 2 hours or 30 minutes respectively. Cells were then washed, stained with a PE-labelled anti-MHC-II monoclonal antibody and analysed by FACS. At 37° C. the cells phagocytosed the FITC-labelled latex beads, but not at 4° C., and upregulated MHC Class II. However, while the cells at 37° C. endocytosed the FITC-dextran, but not at 4° C., they did not upregulate MHC Class II much compared to cells at 4° C. with FITC-dextran or cells at 37° C. without either FITC-labelled latex beads or FITC-dextran. Classic dendritic cells would upregulate MHC Class II if they endocytosed the FITC-dextran at 37° C. which further shows that the dendritic cells of the invention cannot mature.

5.3) Lack of Maturation

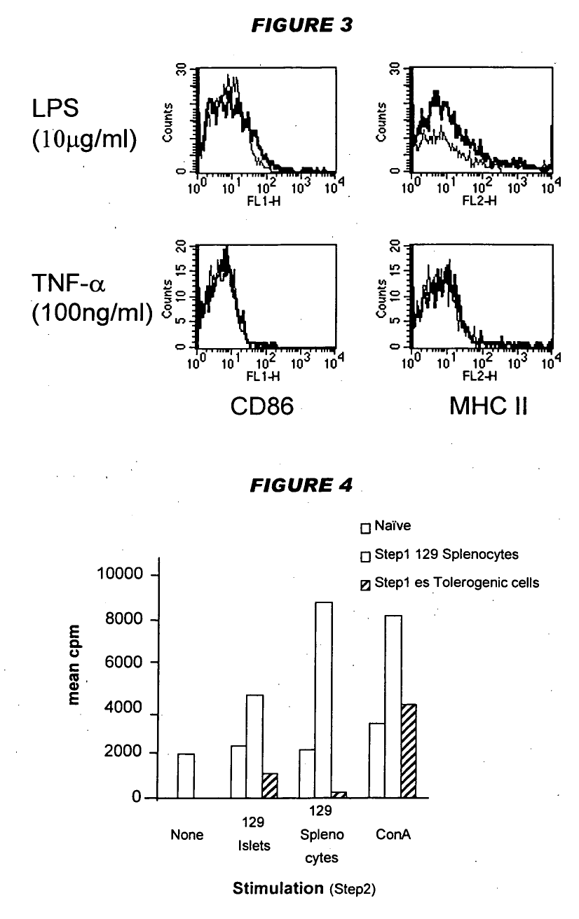

Further evidence that the dendritic cells of the invention cannot mature is the fact that they can not be induced to mature in the presence of even high concentrations of LPS (1-100 μg/ml), TNFα (25-200 ng/ml), PHA (1-100 μg/ml), or ConA (1-100 μg/ml). The cells were prepared from EBs as described above and cultured for 24 or 48 hours in Complete RPMI with or without the aforementioned maturation inducers. Under these conditions these cells did not up-regulate MHC-II or co-stimulatory molecule B7-1 and B7-2 (FIG. 3). The cells of the invention thus stay in an immature state in the presence of inflammatory mediators. Also, after 5 days in the presence of allogeneic T cells (e.g. from CBA/Ca mice which are H-2^{k}) that were purified by StemSep™ columns using their murine T cell purification cocktail the cells of the invention remain in an immature state. This behaviour indicates that they are stable tolerogenic cells which can be used for in vivo tolerance strategies.

5.4) Induction of Tolerance

The cells of the invention can be used in vitro to induce allogeneic T cells to be tolerant towards other cells of the same haplotype (H-2^{b}) as the tolerogenic cells. Dendritic cells were prepared from EBs as described above and cultured for 24 hours in tissue culture flasks in Complete RPMI. During this time the dendritic cells adhere to the plastic. Allogeneic T cells (e.g. from CBA/Ca mice, which are H-2^{k}) were purified by StemSep™ columns using their murine T cell purification cocktail and were then added to the flask of dendritic cells for 7 days. The allogeneic T cells were washed from the dendritic cells, rested overnight, and put in vitro with splenocytes or pancreatic islets from 129/sfv mice which are of the same haplotype (H-2^{b}) as the dendritic cells. At day 6, plates were pulsed with ³H-thymidine and harvested on day 7 to assess levels of proliferation.

CBA/Ca T cells that were previously exposed to dendritic cells of the invention for 7 days hardly proliferated compared with T cells that were either not previously exposed to any cell with the same haplotype as the dendritic cells, or with CBA/Ca T cells that have been exposed to splenocytes with the same haplotype as the dendritic cells (FIG. 4). The T cells in the H-2^{k} recipient would normally attack the H-2^{b} graft, but the H-2^{b} dendritic cells were able to prevent this. The cells of the invention are thus tolerogenic and are able to induce antigen-specific tolerance.

Proof that the CBA/Ca T cells exposed to the tolerogenic cells in the primary culture are not merely made unresponsive, regardless of their antigen specificity, is that they can still proliferate in response to a mitogen (ConA) at least as well as naïve CBA/Ca T cells that have never been exposed to the tolerogenic cells. This indicates that the induced tolerance is antigen-specific and thus will leave the host's immune system intact e.g. to fight infection or cancerous cells.

5.5) In vivo Immunogenicity

Cells of the invention were harvested from culture at days 20 to 35 and injected intravenously into recipient mice having a different haplotype (H-2^{k}) from the ES-derived cells (H-2^{b}). This difference in haplotype would be expected to provoke an immune response in the recipient mice.

As a control, similar H-2^{k} mice were injected with spleen cells from H-2^{b} mice. Again, the difference in haplotype would be expected to provoke an immune response. As a further control, another group of mice received no injected cells.

At various time intervals after time zero (injection of cells), spleens were removed from the mice and splenocytes were isolated. These cells contain representatives of all the major immune cells of the mouse. These cells were cultured with spleen cells from H-2^{b} mice to see what type of response the injected cells had provoked (the recall response). Results were as follows:

| Injected cells | IFN-γ (pg/ml) | IL-10 (pg/ml) |
|---|---|---|
| None | 95.1 | 67.9 |
| H-2^{b} spleen cells (8 days) | 297.2 | 181.4 |
| H-2^{b} spleen cells (30 days) | 394.8 | 202.1 |
| ES-derived cells (8 days) | 687.6 | 386.8 |
| ES-derived cells (30 days) | 674 | 623.3 |
| Assay positive control | 76.7 | 720 |
| Assay negative control | 0 | 0 |

Thus the recall response of mice receiving injected spleen cells was predominantly the production of interferon gamma (IFN-γ), which is consistent with a rigorous T cell response to foreign cells. This would be the type of response expected in tissue rejection. However, the recall response of mice which received the cells of the invention was the production of interleukin 10 (IL-10), which is indicative of the presence of regulatory T cells. These would be expected if immunological tolerance had been induced. Furthermore, IL-10 was seen only when the spleen cells were cultured with H-2^{k} cells in vitro, which is indicative of antigen specificity.

Overall, these results suggest that intravenous injection of the cells of the invention, but not of spleen cells, induces a regulatory T cell population indicative of immunological tolerance induction.

6) Generation of Tolerogenic Cells from CBA ES Cells

CBA murine embryonic stem cells were obtained from the CBA mouse strain [81] and were maintained as described above for HM-1 cells. The method for deriving tolerogenic cells from CBA ES cells was similar to that used for HM-1 cells. When a T25 flask of undifferentiated HM-1 cells were confluent, the cells were trypsinised lightly, so clumps of cells appeared as opposed to all single cells, washed at 900 rpm for 2 minutes to allow clumps of cells to collect at bottom of tube, supernatant carefully removed and clumps gently resuspended in 5 ml Complete Medium without LIF. 1.5-2×10⁵ cells/cell clumps were plated onto 90 mm bacteriological plastic dishes in 10 ml Complete Medium. Under these conditions, the CBA ES cells failed to adhere to the bacteriological plastic but remained in suspension where they continued to proliferate and form EBs. These spheres became macroscopic by day 4-7 of culture and adopted a cystic appearance by day 10-14. Cells were kept in incubators at 37° C. with 5% CO₂.

At day 4-7, the EBs were transferred to a universal tube and 60-80 μl were added to each well of 6-well tissue culture plates. 2 ml/well of Complete Medium supplemented with 25 ng/ml recombinant murine GM-CSF as well as or without 1000 U/ml recombinant murine IL-3 was added. Cells were kept in incubators at 37° C. with 5% CO₂.

Within 24 hours of culture the EBs adhered to the plastic and growth of differentiating cells, mainly stromal cells, emigrating outwards in a radial fashion appeared. Clusters of tolerogenic cells start to appeared by day 10-14 and by day 21 the clusters were large enough to harvest the tolerogenic cells. Some of the tolerogenic cells adhered strongly to the plastic but most of them lightly adhered to the underlying layer of cells. They could be harvested by gentle pipetting and passaged over a 70 μm cell strainer to remove unwanted debris. Since the stromal layer which supports the generation of the tolerogenic cells is left intact, repeated harvesting of tolerogenic cells could be continued for 4 to 5 weeks.

7) Characterization/Phenotype of CBA ES Cell-Derived Tolerogenic Cells

The CBA ES-cell-derived tolerogenic cells were analysed by flow cytometry for expression of surface markers using a panel of monoclonal antibodies to determine their phenotype. CD11b, CD54 (ICAM-1), and F4/80 were expressed on the surface of the tolerogenic cells. Low or insignificant expression of CD11c and MHC-II was observed on the tolerogenic cells.

It will be understood that the invention is described above by way of example only and modifications may be made whilst remaining within the scope and spirit of the invention.

### REFERENCES

[1] Waldmann et al. (2001) Int Arch Allergy Immunol. 126: 11-22.
[2] Kingsley et al. (2002) J. Immunol. 168: 1080-1086.
[3] Grohmann et al. (2001) J. Immunol. 166: 277-283.
[4] Zhou et al. (2001) Transplant Proc. 33: 214-216.
[5] O-Connell et al. (2002) J. Immunol. 168: 143-154.
[6] Fong et al. (2001) J. Immunol. 167: 7150-7156.
[7] Nenciomi et al. (2001) BioDrugs. 15:667-679.
[8] Morelli et al. (2001) Semin Immunol. 13: 323-335.
[9] Lechler et al. (2001) Immunity 14: 357-368.
[10] Mellman et al. (2001) Cell 106: 255-258.
[11] U.S. Pat. Nos. 5,871,728 and 6,224,859.
[12] Pulendran et al. (1999) PNAS USA 96:1036-1041.
[13] Liu (2001) Cell 106: 259-262.
[14] Maldonado-Lopez et al. (1999) J. Exp. Med. 189: 587-592.
[15] WO 01/85920.
[16] Mohamadzadeh et al. (2001) J. Exp Med. 194:1013-1020.
[17] WO00/28000; see also US patent application publication 20020019047.
[18] Fairchild et al. (2000) Current Biology 10:1515-1518.
[19] WO01/02005.
[20] Urban et al. (2001) PNAS USA 98:8750-8755.
[21] WO01/83713.
[22] WO00/61132.
[23] Thomas et al. (2000) Transplantation 69:2497-2503.
[24] WO01/09288.
[25] Tarte et al. (2000) Leukemia 14:2182-2192.
[26] U.S. Pat. No. 6,090,622.
[27] U.S. Pat. No. 5,670,372.
[28] U.S. Pat. No. 6,200,806.
[29] Thomson et al. (1998) Science 282, 1145-7.
[30] Smith (2001) Ann. Rev Cell Dev Biol 17:435-62.
[31] National Institutes of Health, Dept of Health and Human Services Report. Stem cells: scientific progress and future research directions. June 2001. www.nih.gov/news/stemcell/scireport.htm.
[32] Robertson (1987) Teratocarcinomas and embryonic stem cells, a practical approach. IRL Press.
[33] Wobus (2001) Mol Aspects Med 22:149-64.
[34] Tessarollo (2001) Methods Mol Biol 158:47-63.
[35] Wobus et al. (2001) Methods Mol Biol 158:263-86.
[36] Marshall et al. (2001) Methods Mol Biol 158:11-18.
[37] Wobus et al. (2000) Cells Tissues Organs 166:1-5.
[38] Pera et al. (2000) J Cell Sci 113:5-10.
[39] Lebkowski et al. (2001) Cancer J7 Suppl 2:S83-93.
[40] Embryonic Stem Cells: Methods and Protocols (ed. Turksen) 2002. ISBN 0896038815.
[41] Xu et al. (2002) Nat Biotechnol 20:1261-1264.
[42] Zhang et al. (2001) Nature Biotechnol. 19:1129-1133.
[43] Donovan et al. (2001) Nature 414:92-97.
[44] Pera (2001) Curr Opin Genet Dev. 11:595-599.
[45] Muench et al. (2001) J Immunol. 167:49024909.
[46] Foley et al. (2001) Transfus Med Rev. 15:292-304.
[47] Oki (2001) Exp Hematol. 29:1210-1217.
[48] Richter et al, (2001) J Biol Chem. 276:4568645693.
[49] Wu et al. (2001) Int Rev Immunol. 20:117-135.
[50] Reubinoffet al. (2000) Nature Biotech 18:399404.
[51] Desbaillets et al. (2000) Exp Physiol 85:645-51.
[52] Keller (1995) Curr Opin Cell Biol 7:862-9.
[53] Mueller-Klieser (1997) Am J Physiol 273:C1109-23.
[54] Magyar et al. (2001) Ann N Y Acad Sci 944:13543.
[55] Schuldiner et al. (2000) PNAS USA 97:11307-11312.
[56] Carpenter et al. (2001) Exp Neurol 172(2):383-397.
[57] Schuldiner et al. (2001) Brain Res 913(2):201-205.
[58] Kehat et al. (2001) J Clin Invest 108(3):407414.
[59] Eiges et al. (2001) Curr Biol 11:514-518.
[60] Schuh & Morrissey (1999) Toxicol Pathol 27:72-77.
[61] Manual of GM-CSF. (ed. Marty). ISBN: 0865428182.
[62] Ly et al. (2001) Mol Pathol 54(4):230-9.
[63] Qian et al. (2001) Transplant Proc 33:551.
[64] Liang et al. (2001) Transplant Proc 33:235.
[65] Liu et al. (2000) Cancer Gene Ther 7(3):456-65.
[66] Zamore (2001) Nat Struct Biol 8:746-750.
[67] Carthew (2001) Curr Opin Cell Biol 13:244-248.
[68] Billy et al. (2001) PNAS USA 98:14428-14433.
[69] Yang et al. (2001) Mol Cell Biol 21:7807-7816.
[70] Doty & Clark (1998) J Immunol 161:2700-7.
[71] Goldstein & Watts (1996) J Immunol 157:2837-43.
[72] Hostager et al. (1996) J Immunol 157:1047-53.
[73] Rubanyi (2001) Mol Aspects Med. 22:113-142.
[74] Springer & Niculescu-Duvaz (2000) J Clin Invest 105:1161-7.
[75] Niwa et al. (2000) Nature Genet 4:372-6.
[76] Suzuki et al. (1990) EMBO J 11:3723-32.
[77] Ben-Shushan et al. (1998) Mol Cell Biol 18:1866-78.
[78] Sutton et al. (1996) J Biol Chem. 271:23126-33.
[79] Ledermann (2000) Exp Physiol 85:603-13.
[80] Magin et al. (1992) Nucleic Acid Res. 20:3795-3796.
[81] McWhir et al. (1996) Nature Genetics 14:223-226.

1. A dendritic cell which is immature and cannot mature.

2. A dendritic cell which is able to present antigens to T cells, which is CD40^{-ve} CD80^{-ve} and CD86^{-ve}, and which remains CD40^{-ve} CD80^{-ve} and CD86^{-ve} when stimulated by inflammatory mediators.

3. A dendritic cell which can deliver signal 1 to a T cell, but which cannot provide signal 2 to the T cell, either in a resting state or when stimulated by an inflammatory mediator.

4. A tolerogenic dendritic cell differentiated in vitro from an ES cell.

5. The cell of any one of claims 1 to 4, wherein the cell is not immortal.

6. The cell of any one of claims 1 to 4, wherein the cell has a normal karyotype.

7. The cell of any one of claims 1 to 4, wherein the cell is a human cell.

8. A cell obtainable by the method of any one of claims 9 to 15.

9. A process for preparing a tolerogenic antigen-presenting cell from a stem cell, wherein the method includes the step of culturing the stem cell in the presence of one or more cytokine(s) which cause(s) the stem cell to differentiate into the tolerogenic cell.

10. The process of claim 9, wherein the stem cell is an embryonic stem cell.

11. The process of claim 9 or claim 10, wherein the stem cell is a human stem cell.

12. The process of any one of claims 9 to 11, wherein the stem cells develop into embryoid bodies before differentiation into the tolerogenic cells.

13. The process of any one of claims 9 to 12, wherein differentiation into the tolerogenic cell takes place in adherent culture.

14. The process of any one of claims 9 to 13, wherein a feeder layer is not used.

15. The process of any one of claims 9 to 14, wherein the cytokine is GM-CSF.

16. The cells of any one of claims 1 to 4 for use as a medicament.

17. The use of the cells of any one of claims 1 to 4 in the manufacture of a medicament for inhibiting an autoimmune reaction.

18. The use of the cells of any one of claims 1 to 4 in the manufacture of a medicament for inhibiting graft rejection in a recipient.

19. A method of inhibiting graft rejection in a recipient, wherein the cells of any one of claims 1 to 4 are administered to the recipient.

20. A method for transplanting a graft into a recipient, wherein the method also involves the administration of the cells of any one of claims 1 to 4 to the recipient.

21. The method or use of any one of claims 18 to 20, wherein the graft is heart, lung, kidney, liver, pancreas, islets of Langerhans, pancreatic β-cells or other insulin-producing cells, cornea, bone marrow or nervous tissue.

22. The method or use of any one of claims 18 to 20, wherein the dendritic cells are histocompatible with the graft.

23. A method of inhibiting an autoimmune reaction in a patient, wherein the cells of any one of claims 1 to 4 are administered to the patient.

24. A kit comprising (a) the cells of any one of claims 1 to 4 and (b) a tissue graft for transplanting into a recipient, wherein (a) and (b) are histocompatible.

25. A composition comprising the cells of any one of claims 1 to 4 and a pharmaceutical carrier.

26. A stem cell for use in the process of any one of claims 9 to 15, wherein the stem cell has been genetically manipulated.

27. The stem cell of claim 26, wherein the stem cell has been genetically manipulated to encode a polypeptide which promotes differentiation of the stem cell into a dendritic cell.

28. The stem cell of claim 26, wherein the stem cell has been genetically manipulated to express or over-express one or more surface proteins which down-regulate immune responses.

29. The stem cell of claim 26, wherein the stem cell has been genetically manipulated not to express or to under-express surface and/or secreted proteins which promote T cell activation.

30. The stem cell of claim 26, wherein the stem cell has been genetically manipulated to include a suicide gene.

31. The stem cell of claim 26, wherein the stem cell has been genetically manipulated to include a marker suitable for lineage selection.
